(19) United States
(12) Patent Application Publication
(10) Pub. No.: US 2024/0390554 A1
(43) Pub. Date: Nov. 28, 2024

(54) DECELLULARIZED MUSCLE MATRIX

(71) Applicant: LifeCell Corporation, North Chicago, IL (US)

(72) Inventors: Hui Xu, Plainsboro, NJ (US); Eric Stec, Washington, NJ (US); Li Ting Huang, Branchburg, NJ (US)

(21) Appl. No.: 18/583,308

(22) Filed: Feb. 21, 2024

Related U.S. Application Data
(63) Continuation of application No. 16/598,597, filed on Oct. 10, 2019, now abandoned.
(60) Provisional application No. 62/854,647, filed on May 30, 2019.
(60) Provisional application No. 62/744,204, filed on Oct. 11, 2018.

(51) Int. Cl. A61L 27/36; A61L 27/38; C12N 5/071

(52) U.S. Cl. CPC A61L 27/3683; A61L 27/3633; A61L 27/367; A61L 27/3826; A61L 27/3873; C12N 5/0697; A61L 2300/64; A61L 2400/06; A61L 2400/18; A61L 2430/30; A61L 2430/40

(57) ABSTRACT

Disclosed herein are muscle implants and methods of making muscle implants comprising one or more decellularized muscle matrices. The muscle matrices can be provided in a particulate form suitable for injection or implantation.

This application is a continuation of U.S. patent application Ser. No. 16/598,597, filed on Oct. 10, 2019, which claims the benefit under 35 U.S.C. § 119 of U.S. Provisional Patent Application No. 62/854,647, filed on May 30, 2019, and U.S. Provisional Patent Application No. 62/744,204, filed on Oct. 11, 2018. The entire contents of each aforementioned provisional application are incorporated herein by reference.

The present disclosure relates generally to methods of making and using decellularized muscle matrices in the repair, regeneration, and/or treatment of abdominal wall and other muscle defects.

Various injuries, diseases, and surgical procedures result in the loss of muscle mass, particularly skeletal muscle. For example, surgical removal of soft tissue sarcomas and osteosarcomas can result in the loss of bulk muscle. Other surgical and cosmetic procedures, such as hernia repair and muscle augmentation, require long-term management of muscle content. Muscle damage can also result from injury, such as from blunt force trauma and gunshot injuries.

Current muscle regenerative procedures focus on the use of muscle allografts (e.g., harvesting gluteus maximus muscle from donor sites on the patient or from a cadaver) or the use of xenografts comprising completely decellularized dermal and other tissue matrices. However, the use of muscle transplants can lead to excess inflammation (resulting in scar tissue formation and potential rejection) and, if harvested from the patient, presents the problem of muscle loss at the donor site. Thus, a need remains for improved methods and compositions for the long-term management of muscle repair and regeneration, including generation of functional muscle mass. Furthermore, there remains a desire for methods of increasing muscle mass, e.g, to improve functional characteristics or aesthetics (e.g., for individuals with low muscle mass or those desiring increased strength or mass).

### SUMMARY

Accordingly, disclosed herein are muscle implants comprising decellularized muscle matrices that retain at least some of the myofibers or other muscle structural proteins normally found in a muscle tissue prior to processing, and their use to improve muscle repair, treatment, enhancement, augmentation, and/or regeneration. In various embodiments, a method of preparing a muscle implant is provided. The method can comprise providing at least one muscle sample; contacting the at least one muscle sample with an enzyme; decellularizing the at least one muscle sample to produce at least one decellularized muscle matrix; and controlling the exposure duration and/or concentration of the enzyme and decellularizing process in order to retain at least some of the myofibers normally found in the muscle sample prior to decellularization.

Also provided are sheet tissue products that provide improved strength. The sheet products can include a decellularized muscle matrix and a decellularized dermal matrix. The matrices can be layered to allow formation of a composite. Methods of using such matrices can allow treatment of complex injuries that require generation of functional muscle. The dermal matrix can provide structural support for load-bearing during muscle regeneration and can provide a substrate for regeneration of connective tissue around or near new muscle.

In various embodiments, a muscle implant is provided, comprising particulate or sheet decellularized muscle matrix. The matrix can contain at least some of the myofibers normally found in a muscle tissue prior to processing. Alternatively or additionally, the muscle can be characterized as maintaining a certain percentage of myosin. In addition, the muscle, although retaining myofibers and/or myosin, can be decellularized as measured by histological staining. In some embodiments, the decellularized muscle matrix contains no more than about 20-80% of the myofibers or myosin normally found in a muscle tissue prior to processing. In certain embodiments, the muscle implant is in particulate form. In certain embodiments, the muscle implant is lyophilized or provided in aqueous solution.

In various embodiments, a method of treatment is provided, comprising injecting or implanting into a patient one of the muscle implants described above. In some embodiments, the muscle implant promotes an increased rate and/or overall amount of native muscle regeneration after implantation into a patient, as compared to the rate and/or overall amount of regeneration in the absence of an implant or in the presence of an implant comprising intact muscle or comprising decellularized muscle that lacks substantially all myofibers or myosin. In certain embodiments, the muscle implant is used to treat a skeletal muscle defect such as an abdominal hernia, abdominal injury, surgical insult, gunshot wound, or blunt force trauma. In some embodiments, the muscle implant is used after the loss of bulk muscle, for example, due to a muscle wasting disorder or due to the surgical removal of native muscle tissue from a patient (e.g., from a treatment of a sarcoma or osteosarcoma). In certain embodiments, the muscle implant is used to enhance the appearance and/or volume of muscle tissue at an implant site. The muscle implant can be provided with one or multiple injections of particulate muscle matrix. The muscle implants can be used to provide functional muscle regeneration or augmentation as measured by contractile force.

### DESCRIPTION OF THE DRAWINGS

FIG. 1 is a flow chart illustrating an exemplary method for producing the disclosed muscle matrices.

FIGS. 2A and 2B are histological images of fresh porcine muscle and porcine acellular muscle matrix, as prepared according to various disclosed embodiments.

FIG. 3 is a bar graph showing myosin content of fresh porcine muscle versus that of porcine acellular muscle matrix (pAMM) in either sheet or particulate form.

FIGS. 4A and 4B are trichome-stained sections of rat rectus abdominal defects six weeks after creation and treatment with porcine acellular dermal matrix or a combination of porcine acellular dermal matrix and porcine acellular muscle matrix.

FIG. 5 is a graph of functional muscle recovery as measured by running distance for rats with gastrocnemius muscle defects left untreated or repaired with pAMM.

FIG. 6 is a bar graph of functional muscle recovery as measured by contractile force for rats with gastrocnemius muscle defects left untreated or repaired with pAMM.

FIGS. 7A and 7B provide trichrome-stained images of rat tibialis anterior (TA) muscle defects three weeks after defect creation. Defects were either left unrepaired (A) or repaired with injectable pAMM (B) immediately after defect creation.

FIG. 8 is a bar graph illustrating TA muscle weights three weeks after defect creation with or without repair using injectable pAMM, and compared to a contralateral muscle.

FIGS. 9A and 9B are trichrome-stained images of primate gastrocnemius defects twelve weeks after defect creation. Defects were either left unrepaired (A) or repaired with pAMM (B) immediately after defect creation.

FIGS. 10A and 10B provide gross cross-sectional images of rat TA muscles injected with pAMM at one or three weeks after injection.

FIG. 11 is an H&E section of rat TA muscle injected with pAMM three weeks after implantation and demonstrating new muscle formation.

FIG. 12 is a bar graph of rat TA muscles with or without pAMM injection three weeks after treatment to show pAMM ability to augment existing muscle mass.

FIG. 13 is a bar graph of rat TA muscles injected with pAMM during 1, 2, or 3 treatments at nine weeks after injection.

FIG. 14 illustrates the percentage of weight increase of injected muscle compared to their corresponding contralateral muscle, as described in Example 8.

FIG. 15 provides Trichrome stained images of muscles injected with various formulations of pAMM at 3 weeks post injection, as described in Example 8. No residual amounts of Formulation 1 pAMM are detectable while small amounts of Formulation 2 and 3 pAMM are detectable.

FIG. 16 provides trichrome stained images of muscles injected with various formulations of pAMM at 6 weeks post injection, as described in Example 8. No residual amounts of Formulation 1 or Formulation 2 pAMM are detectable while a small amount of Formulation 3 pAMM are detectable.

FIGS. 17A-17E provide gross images of trypsin treated porcine muscle tissue.

FIG. 18 is a bar graph of remaining myosin in porcine muscle tissue treated with varying concentrations of trypsin.

FIGS. 19A-19J provide gross images of trypsin treated, bromelain treated, and untreated porcine muscle tissue.

FIG. 20 is a bar graph of remaining myosin in porcine muscle tissue treated with varying concentrations of bromelain.

### DESCRIPTION OF CERTAIN EXEMPLARY EMBODIMENTS

Reference will now be made in detail to certain exemplary embodiments according to the present disclosure, certain examples of which are illustrated in the accompanying drawings.

As used herein, “myofibers” are the rod-like structures involved in muscle contraction and comprise proteins such as myosin, troponin, tropomyosin, and actinin. Long myofiber chains are found in and between the elongated muscle cells (myocytes).

As used herein, a “muscle defect” is any muscle abnormality or damage that is amenable to repair, improvement, enhancement, regeneration, amelioration, and/or treatment by an implanted muscle matrix. A muscle defect encompasses any abnormality or damage resulting from disease, trauma, or surgical intervention that results in an alteration to the muscle. As used herein, the removal or loss of “bulk” muscle tissue refers to the loss of an appreciable and measurable volume of muscle tissue, e.g., a volume of at least about 0.5 cm³.

As used herein, a “decellularized tissue” is any tissue from which most or all of the cells that are normally found growing in the extracellular matrix of the tissue have been removed (e.g., a tissue lacking about 80, 85, 90, 95, 99, 99.5, or 100% of the native cells) (or any percentage in between). The cell removal can be assessed by light microscopy with H&E sections.

As used herein, the terms “native cells” and “native tissue” mean the cells and tissue present in the recipient tissue/organ prior to the implantation of a muscle implant, or the cells or tissue produced by the host animal after implantation.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. To the extent publications and patents or patent applications incorporated by reference contradict the invention contained in the specification, the specification will supersede any contradictory material.

In this application, the use of the singular includes the plural unless specifically stated otherwise. Also in this application, the use of “or” means “and/or” unless stated otherwise. Furthermore, the use of the term “including,” as well as other forms, such as “includes” and “included,” are not limiting. Any range described here will be understood to include the endpoints and all values between the endpoints.

Disclosed herein are muscle implants comprising one or more decellularized muscle matrices. The muscle matrices can include particulate matrices that are suitable for injection. Injection can be used to treat a number of anatomic sites, including large muscle (e.g., limb, abdominal, neck, torso), or smaller muscles including sphincters, facial muscle, tongue, or hand. The matrices can alternatively include sheets, or sheets with a combination of decellularized muscle and decellularized dermis, or muscle and other materials such as polypropylene mesh. The matrices can be implanted and induce functional muscle regeneration or augmentation.

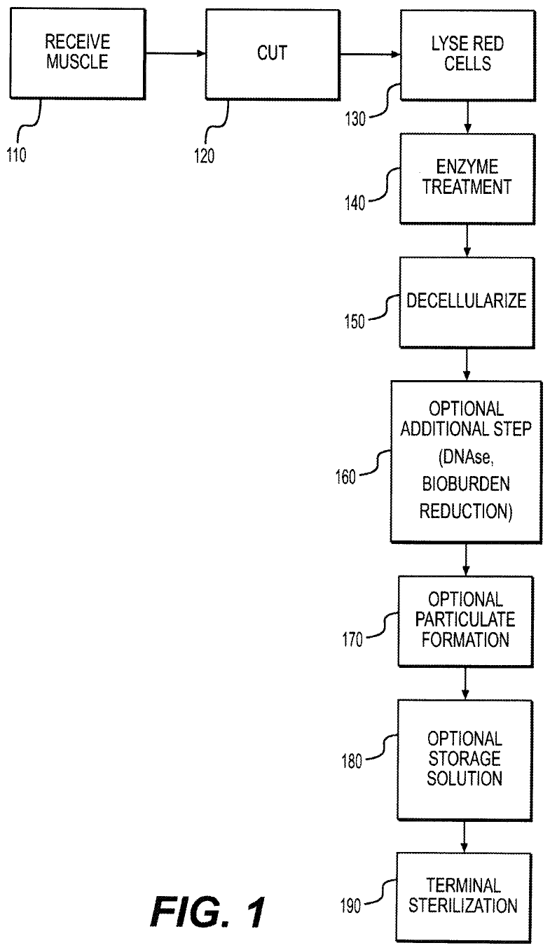

The disclosed muscle matrices can be produced using variations of an exemplary process illustrated in a flow chart of FIG. 1. As shown, the process generally includes, obtaining a muscle (Step 110), cutting the muscle to a desired size (Step 120), and optionally performing a step to lyse or destroy certain cells, including red blood cells (Step 130). The method can further include treating the tissue with an enzyme such as trypsin (Step 140), decellularizing the tissue (Step 150), and optionally performing additional steps to remove or disrupt components of the tissue (Step 160). Next, the tissue can be processed to form particulates (Step 170). The tissue may then be prepared for final storage and sterilization placement in a protective or storage solution (Step 180), followed by terminal sterilization (Step 190). Details of these steps are provided below.

Step 110 includes receiving or obtaining a muscle tissue. The tissue can include skeletal muscle obtained from a number of different animals or anatomic sites on an animal. Alternatively, the tissue could include smooth or cardiac muscle.

The tissue can be obtained from humans or non-human mammals. Furthermore, the muscle can include any suitable muscle, but will often be selected to provide suitable volume to allow for efficient processing. Suitable muscles can include, for example, animal leg, arm, or torso muscles, including, for example, loin, rectus, back, tibialis, or similar muscles.

While the disclosed muscle matrices may be derived from one or more donor animals of the same species as the intended recipient animal, this is not necessarily the case. Thus, for example, the decellularized muscle tissue may be prepared from porcine tissue and implanted in a human patient. Species that can serve as donors and/or recipients of decellularized muscle tissue include, without limitation, mammals, such as humans, nonhuman primates (e.g., monkeys, baboons, or chimpanzees), pigs, cows, horses, goats, sheep, dogs, cats, rabbits, guinea pigs, gerbils, hamsters, rats, or mice. In some embodiments, muscle tissue from more than one donor animal can be used.

In certain embodiments, animals that have been genetically modified to lack one or more antigenic epitopes may be selected as the tissue source for a muscle matrix. For example, animals (e.g., pigs) that have been genetically engineered to lack expression of the terminal α-galactose moiety can be selected as the tissue source. For descriptions of appropriate animals and methods of producing transgenic animals for xenotransplantation, see U.S. Pat. No. 8,802,920, which is titled “Acellular Tissue Matrices Made from Alpha-1,3-Galactose Deficient Tissue,” which granted on Aug. 12, 2014 and which is hereby incorporated by reference in its entirety. Alternatively, tissue can be treated to remove terminal α-galactose moieties, e.g., by treatment with alpha-galactosidase.

After obtaining muscle but before use, the tissue can be stored to prevent damage or undesirable changes. For example, the tissue can be frozen at cryogenic temperatures and slowly thawed to prevent freeze-thaw cycle damage. For example, the tissue may be frozen at −60° C. and thawed when desired over 6-12 hours.

After obtaining the muscle, the muscle can be cut to a desired size to facilitate further processing (Step 120). For example, to allow processing with various solutions, the muscle may be cut into pieces or sheets with a desired thickness or size. A suitable size for cutting can include strips or sheets of about 0.5 mm thickness, but smaller or larger pieces can be used.

After initial cutting, the tissue sample can be processed to remove blood or blood components such as red blood cells (“RBC”) (Step 130). For example, the tissue samples can be exposed to a cell lysis solution to remove cells such as red blood cells. A variety of blood cell removal or lysis solutions can be used, including, for example, solutions such as ammonium chloride, hypo- or hypertonic-saline, detergents, or other know blood removal compositions. Further, the solutions can be used in a number of incubation and/or wash steps, including for example, one to ten wash steps, or any suitable number in between. The tissue can be rinsed to remove lysed RBC material.

After blood lysis, the tissue sample can be contacted with a solution including an enzyme in order to break down muscle fiber bundles (e.g., by cleaving myosin molecules in the muscle fiber) and/or to remove other non-desired components (Step 140). For example, the enzyme can include one or more proteases, such as serine proteases, and the protease can assist in cell removal or disruption, removal of denatured or damaged collagen fragments, or removal of certain antigens such as alpha-galactose.

In some embodiments, the solution can include enzymes such as trypsin or serine proteases. Suitable enzymes can include, for example, papain, bromelain, ficin, or alcalase. In some embodiments, trypsin or other enzymes listed above can facilitate the decellularization process by increasing the rate and/or extent of myofiber breakdown and myocyte removal during subsequent decellularization.

In some embodiments, the muscle sample can be exposed to trypsin at a concentration in a range from about 10⁻¹⁰-0.5% (e.g., at about 0.02, 0.03, 0.04, 0.05, 0.06, 0.07, 0.08, 0.09, 0.1, 0.15, 0.2, 0.25, 0.3, 0.35, 0.4, 0.45, or 0.5 percent), or from 10⁻⁸-10⁻⁴%, or from 10⁻⁷-10⁻⁵%, or from 10⁻⁴-10⁻²%, or any percent in between. The aforementioned concentrations can be considered appropriate for trypsins that have an enzymatic activity such that 10⁻⁶% corresponds to approximately 120-130 BAEE units, and a BAEE unit is determined for enzymes with a specification for trypsin activity using Nα-Benzoyl-L-arginine ethyl ester (BAEE) as a substrate. The procedure is a continuous spectrophotometric rate determination (ΔA253, Light path=1 cm) based on the following reaction:

$$BAEE + {H}_{2} O {\to }^{trypsin} N α - Benzoyl - L - arginine + ethanol where :$$

$$BAEE = N α - Benzoyl - L - arginine ethyl ester ; and$$

A BAEE Unit is defined such that one BAEE unit of trypsin activity will produce a ΔA253 of 0.001 per minute with BAEE as substrate at pH 7.6 at 25° C. in a reaction volume of 3.20 ml.

A number of suitable trypsins may be used, but one exemplary trypsin that may be appropriate includes bovine pancreatic trypsin, human pancreatic trypsin, porcine pancreatic trypsin, recombinant human trypsin, and recombinant porcine trypsin.

In some embodiments, the muscle sample can be exposed to bromelain at a concentration in a range from about 5 units per liter to 200 units per liter.

In certain embodiments, the muscle sample can be exposed to trypsin or other enzymes for at least about 15 minutes or up to a maximum of about 24 hours (e.g., about 15 minutes, 30 minutes, 45 minutes, 60 minutes, 75 minutes, 90 minutes, 105 minutes, 120 minutes, 4 hours, 8 hours, 12 hours, 24 hours or any intermediate time). In certain embodiments, muscle samples can be exposed to enzyme for at least about 15 minutes or up to a maximum of about 48 hours (e.g., about 15 minutes, 30 minutes, 45 minutes, 60 minutes, 75 minutes, 90 minutes, 105 minutes, 120 minutes, 4 hours, 8 hours, 12 hours, 24 hours, 48 hours or any intermediate time). In various embodiments, decellularization can be done before trypsinization (or other enzyme treatments), after trypsinization, or both before and after trypsinization.

After enzyme treatment, the tissue sample can be processed to produce a decellularized matrix. As discussed herein, “decellularized” tissue will be understood to refer to muscle matrix with substantially all cells removed as determined by light microscopy. However, as discussed herein, the muscle matrices can retain contractile proteins, including myosin, which has been found to be important in allowing growth of new muscle tissue. Although, myosin and other proteins may be contained within the cells, the “decellularized” or “acellular” muscle matrices referred to herein will be understood to be decellularized or acellular as long as the tissue is visually free of cells on hematoxylin and eosin light microscopy.

The tissue sample can be exposed to a decellularization solution in order to remove viable and non-viable cells from the muscle tissue without damaging the biological and/or structural integrity of an extracellular matrix within the muscle tissue. The decellularization solution may contain an appropriate buffer, salt, an antibiotic, one or more detergents (e.g., TRITON X-100™ or other nonionic octylphenol ethoxylate surfactants, sodium dodecyl sulfate (SDS), sodium deoxycholate, or polyoxyethylene (20) sorbitan monolaurate), one or more agents to prevent cross-linking, one or more protease inhibitors, and/or one or more enzymes. In some embodiments, the decellularization solution can comprise 0.1%, 0.2%, 0.3%, 0.4%, 0.5%, 1.0%, 1.5%, 2.0%, 2.5%, 3.0%, 3.5%, 4.0%, 4.5%, 5.0%, or any intermediate percentage of TRITON X-100™ and, optionally, 10 mM, 15 mM, 20 mM, 25 mM, 30 mM, 35 mM, 40 mM, 45 mM, 50 mM, or any intermediate concentration of EDTA (ethylenediaminetetraacetic acid). In certain embodiments, the decellularization solution can comprise 0.1%, 0.2%, 0.3%, 0.4%, 0.5%, 1.0%, 1.5%, 2.0%, 2.5%, 3.0%, 3.5%, 4.0%, 4.5%, 5.0%, or any intermediate percentage of sodium deoxycholate and, optionally, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, or 20 mM HEPES buffer (4-(2-hydroxyethyl)-1-piperazineethanesulfonic acid) containing 10 mM, 15 mM, 20 mM, 25 mM, 30 mM, 35 mM, 40 mM, 45 mM, 50 mM, or any intermediate concentrations of EDTA. In some embodiments, the muscle tissue can be incubated in the decellularization solution at 20, 21, 22, 23, 24, 25, 30, 31, 32, 33, 34, 35, 36, 37, 38, 39, 40, 41, or 42 degrees Celsius (or any temperature in between), and optionally, gentle shaking can be applied at 10, 20, 30, 40, 50, 60, 70, 80, 90, 100, 110, 120, 130, 140, or 150 rpm (or any rpm in between). The incubation can be for 1, 2, 3, 4, 5, 6, 7, 8, 9, 10, 11, 12, 15, 20, 24, 36, 48, 60, 72, 84, or 96 hours (or any time period in between).

The length of time of exposure to the decellularization solution and/or the concentration of detergent or other decellularizing agents can be adjusted in order to control the extent of decellularization and myofiber or myosin removal from the muscle tissue. In certain embodiments, additional detergents may be used to remove cells from the muscle tissue. For example, in some embodiments, sodium deoxycholate, SDS, and/or TRITON X-100™ can be used to decellularize and separate undesired tissue components from the extracellular tissue matrix.

The procedure to decellularize the tissue sample can, in some embodiments, be controlled to retain at least some myofibers normally found in the tissue sample prior to processing. For example, the length of exposure and/or the concentration of the decellularization solution and/or trypsin solution can be adjusted in order to control the extent of myofiber removal. In some embodiments, the duration and/or concentration are selected in order to remove about 20-80% of the myofibers normally found in the muscle tissue prior to trypsinization/other enzyme treatment and decellularization. In certain embodiments, the duration and/or concentration are selected in order to remove about 20, 30, 40, 50, 60, 70, 80, or 90 percent of the myofibers (or any percentage in between). In some embodiments, about 20-80% of the myofibers are removed by exposing the tissue sample to trypsin at a concentration ranging from 10⁻¹⁰-0.5% for 15 minutes to 24 or 48 hours and/or by exposing the muscle tissue sample to about 0.1-2.0% of a decellularization agent (e.g., TRITON X-100™ or other nonionic octylphenol ethoxylate surfactant, sodium dodecyl sulfate, sodium deoxycholate, or polyoxyethylene (20) sorbitan monolaurate) for 0.1-72 hours.

The amount of myofiber can be analyzed in a number of ways. As used herein, the remaining myofiber can be assessed using light microscopy.

Alternatively, rather than retaining myofiber, the muscle matrices described herein can be treated to produce a desired amount of residual myosin. Myosin may correlate with myofiber content, but can be measured directly using Enzyme-Linked Immunosorbent Assay (ELISA). Accordingly, according to various embodiments, the muscle matrices described herein can be treated to have between 10-50% of the myosin found in fresh muscle, between 20-30% of the myosin found in fresh muscle, or to have a specific myosin concentration (e.g., 50-150 μg/ml or 75-150 μg/ml). Such myofiber or myosin content can be obtained with tissue processed to be substantially acellular, as measured by light microscopy.

In other embodiments, the procedure to decellularize the tissue sample while retaining at least some myofibers normally found in the tissue sample prior to processing can be controlled by adjusting the ratio of tissue mass to volume of decellularization or enzyme solution (e.g., the mass of tissue per volume of solution containing trypsin or other enzyme and/or decellularizing agents). In some embodiments, a lower ratio of tissue to volume of solution can increase the efficiency of the myofiber removal process, thus resulting in a decellularized matrix that retains fewer intact myofibers. In other embodiments, a higher ratio of tissue to volume of solution ratio can reduce the efficiency of the myofiber removal process, thus resulting in a decellularized matrix that retains more intact myofibers.

In various embodiments, the extracellular scaffold within a decellularized muscle tissue may include collagen (particularly collagen type I or type III), elastin, myofiber, and/or other fibers, as well as proteoglycans, polysaccharides, and/or growth factors (e.g. IGF, EGF, Ang 2, HGF, FGF, and/or VEGF). The muscle matrix may retain some or all of the extracellular matrix components that are found naturally in a muscle prior to decellularization, or various undesirable components may be removed by chemical, enzymatic, and/or genetic means. In general, the muscle extracellular matrix provides a structural scaffold comprising fibers, proteoglycans, polysaccharides, and growth factors into which native cells and vasculature can migrate, grow, and proliferate after implantation in a patient. The exact structural components of the extracellular matrix will depend on the type of muscle and/or fascia selected and the processes used to prepare the decellularized tissue.

In certain embodiments, the tissue sample including muscle can be chemically treated to stabilize the tissue so as to avoid biochemical and/or structural degradation before, during, or after cell removal. In various embodiments, the stabilizing solution can arrest and prevent osmotic, hypoxic, autolytic, and/or proteolytic degradation; protect against microbial contamination; and/or reduce mechanical damage that can occur during decellularization. The stabilizing solution can contain an appropriate buffer, one or more antioxidants, one or more oncotic agents, one or more antibiotics, one or more protease inhibitors, and/or one or more smooth muscle relaxants. In some embodiments, the stabilizing solution can include one or more free radical scavengers including, but not limited to, glutathione, n-acetylcysteine, superoxide dismutase, catalase, or glutathione peroxidase.

In certain embodiments, a muscle implant can comprise one or more additional agents. In some embodiments, the additional agent(s) can comprise an anti-inflammatory agent, an analgesic, or any other desired therapeutic or beneficial agent. In certain embodiments, the additional agent(s) can comprise at least one added growth or signaling factor (e.g., a small cell growth factor, an angiogenic factor, a differentiation factor, a cytokine, a hormone, and/or a chemokine). These additional agents can promote native muscle migration, proliferation, and/or vascularization. In some embodiments, the growth or signaling factor is encoded by a nucleic acid sequence contained within an expression vector. As used herein, the term “expression vector” refers to any nucleic acid construct that is capable of being taken up by a cell, contains a nucleic acid sequence encoding a desired protein, and contains the other necessary nucleic acid sequences (e.g., promoters, enhancers, initiation and termination codons, etc.) to ensure at least minimal expression of the desired protein by the cell.

After decellularization, additional processing steps may be performed (Step 160). In certain embodiments, the matrix can be treated with one or more enzymes to remove undesirable antigens, e.g., an antigen not normally expressed by the recipient animal and thus likely to lead to an immune response and/or rejection. For example, in certain embodiments, muscle tissue can be treated with alpha-galactosidase to remove alpha-galactose (α-gal) moieties. In some embodiments, to enzymatically remove α-gal epitopes, after washing the muscle tissue thoroughly with saline, the tissue may be subjected to one or more enzymatic treatments to remove α-gal antigens, if present in the sample. In certain embodiments, the muscle and/or fascia tissue may be treated with an α-galactosidase enzyme to substantially eliminate α-gal epitopes. In addition, certain exemplary methods of processing tissues to reduce or remove alpha-1,3-galactose moieties are described in Xu et al., “A Porcine-Derived Acellular Dermal Scaffold That Supports Soft Tissue Regeneration: Removal of Terminal Galactose-α-(1,3)-Galactose and Retention of Matrix Structure” Tissue Engineering Part A, Vol. 15 (7), 1807-1819 (2009), which is hereby incorporated by reference in its entirety.

In some embodiments, after decellularization, the muscle tissue is washed thoroughly. Any physiologically compatible solutions can be used for washing. Examples of suitable wash solutions include distilled water, phosphate buffered saline (PBS), or any other biocompatible saline solution. In some embodiments, the wash solution can contain a disinfectant such as a weak acid. In certain embodiments, e.g., when xenogenic or allogenic material is used, the decellularized muscle tissue is treated (e.g., overnight at room temperature) with a deoxyribonuclease (DNase) solution. In some embodiments, the tissue sample is treated with a DNase solution Optionally, an antibiotic solution (e.g., Gentamicin) may be added to the DNase solution. Any suitable DNase buffer and/or antibiotics can be used.

In various embodiments, a muscle implant comprising a particulate decellularized muscle matrix is disclosed. Accordingly, after or before enzyme and decellularization steps, the tissue may be formed into particulates (Step 170). For example, the decellularized muscle matrices described above can be cut, blended, cryofractured, or otherwise homogenized to form particulate matrices that can be lyophilized and stored dry, or stored suspended in a gel, hydrogel, or other aqueous solution. In some embodiments, a particulate decellularized muscle matrix can be used as a flowable and/or injectable composition that can be readily molded to fill an implant site and used to repair a muscle defect, bulk up a weakened muscle tissue, or enhance healthy muscle tissue.

Particulate can be formed using a number of processing steps. For example, suitable methods for producing particulate can include cryomilling, cryogrinding, biopsy punching, a meat grinder, hand chopping, or dry milling. The specific method for particulate formation can be selected to produce a desired size range. For example, the particulate size can be selected to allow injection using standard sized syringes or cannulas. Suitable particles can have sizes ranging from about 3 μm to about 5,000 μm. Further, the particles may be sorted or filtered (e.g., with a sieve) to produce a particles size range. A preferred size range is 100 μm (cryomill method) to 800 μm (cryogrind method). The particle size may be selected based on various factors or intended uses. For example, cosmetic injections may preferentially require larger sizes such as 700-800 μm, while indications that require very small needles (e.g., sphinter injection) may require smaller sizes such as 50-200 μm.

After particle formation or with sheet tissues, the tissue can be prepared for storage and sterilization. For example, as indicated at Step 180, the tissue may be placed in a storage or protective solution. Suitable protective solutions can include aqueous solutions or solutions with cryoprotectants, antibacterials, radiation protective materials, or substances that stabilize the tissue. Suitable storage solutions are described, for example, in U.S. Pat. No. 8,735,054, which issued on May 27, 2014, and is titled “Acellular Tissue Matrix Preservation Solution.”

After placement in a solution, the tissue can be terminally sterilized (Step 190). Sterilization can be performed using chemical sterilization or radiation (e.g., gamma, e-beam, or UV). Suitable sterilization processes are discussed in U.S. Pat. No. 8,735,054, which is referenced above, and which is herein incorporated by reference.

### Methods of Use

In various embodiments, a muscle implant comprising a decellularized muscle matrix that retains at least some myofibers can be implanted into a patient (e.g., to fill a region of bulk muscle loss or to cosmetically enhance a muscle tissue). In some embodiments, the remaining myofibers in the muscle matrix can induce an inflammatory response at the implant site. In some embodiments, the inflammatory response is sufficient to initiate and/or enhance the patient's muscle repair machinery without causing excessive inflammation that could result in increased scar tissue formation and/or implant rejection. In some embodiments, the induction of an inflammatory response initiates and/or enhances muscle repair in the patient, e.g., by recruiting macrophages and myoblasts that infiltrate the muscle matrix, and by activating satellite cells that differentiate into muscle within the scaffold provided by the muscle matrix, thereby remodeling the implant into muscle tissue. In various embodiments, activation of the innate muscle repair machinery increases the extent and/or kinetics of muscle repair/regeneration at the implant site. In contrast, muscle repair in the absence of an implant, or when using an implant comprising normal myofiber content or decellularized tissue lacking any myofibers, results in a slower rate of muscle repair and a lower level of muscle tissue formation (and a concomitant increase in connective and/or scar tissue formation).

In some embodiments, a particulate muscle implant can be used to fill a void space in a muscle tissue. For example, a particulate muscle implant in aqueous solution can be flowed into an implant site, filling a desired space and/or increasing the bulk of a muscle tissue. In some embodiments, a particulate muscle implant can be used to pack the space around a non-particulate muscle implant in order to more fully fill the implant site.

A muscle implant, as disclosed herein, can be used in any surgical procedure where repair, alteration, regeneration, and/or enhancement of muscle tissue is desired. For example, a muscle implant can be used in the repair of abdominal wall defects (e.g., hernia repair, gunshot injury, or other abdominal trauma).

In some embodiments, a muscle implant can also be used after surgical removal of bulk muscle tissue (e.g., after surgical intervention to remove a sarcoma or osteosarcoma). In these embodiments, the muscle implant can initiate and/or improve the rate and overall volume of muscle repair by inducing a sufficient (but not excessive) level of inflammation that serves to recruit the patient's muscle repair pathways (e.g., macrophage/myoblast recruitment and satellite cell activation). In contrast, the rate and overall volume of muscle repair is reduced in patients that do not receive a muscle implant and in patients that receive an implant comprising intact muscle or decellularized tissue that lacks any remaining myofibers. Similarly, in surgical procedures where muscle tissue is harvested from one muscle for transplantation into another location on the patient, a muscle implant as described above can be placed at the harvest site to help promote the rate and overall extent of muscle repair at the harvest site following the transplant procedure.

In some embodiments, a muscle implant can be used to enhance native muscle volume. For example, a muscle implant can be used as part of a treatment for a muscle wasting disease, thereby enhancing the rate of repair and regeneration, and/or increasing the overall volume of muscle at the implant site. In another example, the implant can be used to cosmetically enhance the appearance of muscle tissue by promoting the growth of additional muscle volume at the implant site.

As discussed above, the muscle matrices can be provided in sheet or particulate form. When provided in sheet form, the matrix may be combined with a dermal matrix (e.g., ALLODERM® or STRATTICE), or a biological, synthetic (e.g. polypropylene), biodegradable, or bioresorbable mesh. When combined, the sheet of muscle and dermal material can be layered and attached, e.g., by gluing, suturing, or otherwise connecting.

When implanted, the sheet material can be placed in a muscle defect, either alone or in combination with a dermal matrix, or a biological, synthetic (e.g. polypropylene), biodegradable, or bioresorbable mesh. If a dermal matrix is used, the dermal matrix will provide structural support and can provide a substrate for regeneration of connective tissue. For example, a muscle/dermal material can be placed in an abdominal defect with the muscle abutting the region where functional muscle is desired, and the dermal material can be placed where abdominal fascial layers would normally be present.

Particulate muscle matrix can be implanted in a number of ways. For example, as noted, the particulate can be provided to muscle defects or to enhance or enlarge native muscle. Further, the particulate can be provided as a single injection or injections to a muscle site, or multiple injections can be performed. For example, injections can be administered during subsequent treatments spaced out at various intervals e.g., 1 week, 2 weeks, 3 weeks, 4 weeks, or longer. Additionally, or alternatively, multiple injections can be administered at a single treatment time.

### EXAMPLES

The following examples serve to illustrate, and in no way limit, the present disclosure.

### Example 1: Preparation of Muscle Implants and Mechanical Analysis

Porcine skeletal muscle was dissected and stored frozen until ready to use. The tissue was then washed, cut to 5 mm thick slices or sheets and washed to remove RBCs with ammonium chloride lysis buffer. Muscle samples were treated with trypsin. Samples were then placed in a decellularization solution containing detergent, before being washed in HEPES solution. Samples were treated with DNase to remove any DNA remaining in the tissue, and then treated overnight with alpha-galactosidase to remove alpha gal epitopes on the tissue. Samples were exposed to weak acid, washed, and exposed to e-beam radiation.

The extent of myofiber removal was adjusted by controlling the exposure to trypsin and to the decellularization solution.

The decellularized muscle matrix was then subjected to various size reduction processes to produce particulate matrix suitable for injection. Size reduction was done using cryomilling, cryogrinding, biopsy punching, a meat grinder, or dry milling. Particles can also be formed by hand chopping, e.g., with scalpels.

After size reduction, particle sizes were analyzed. Briefly, 10 mL samples of a 5% solid solution of pAMM in deionized water were prepared and analyzed with a Horiba Particle Analyzer following manufacturer's directions. Three runs were taken per sample and an average was value was determined. Table 1 provides particles size information for each size-reduction method

TABLE 1
Size ranges for injectable particles generated
using various size reduction methods.

| Size Reduction | Minimum Particle | Maximum Particle |
|---|---|---|
| Method | Size (μm) | Size (μm) |
| Cryomill | 4.472 | 1531.914 |
| Cryogrind | 8.816 | 3458.727 |
| Biopsy punch | 29.907 | 4537.433 |
| Meat grinder | 5.867 | 4537.433 |
| Dry mill | 5.867 | 3961.533 |

To assess ease of injectability using different particles, injection force was measured. Briefly, an Instron mechanical test system was set up with a 100N load cell and 50N compression platen. A syringe was loaded onto the base plate, and force generated by compressing the plunger of syringe at 1 mm/sec for 25 mm was measured. Table 2 provides injection force data.

TABLE 2
Maximum force needed to inject a slurry of size reduced pAMM
created using various methods through a 1 mL syringe.

| | Maximum Injection | |
|---|---|---|
| Size Reduction | Force (N) (Average ± | |
| Method | Standard Dev) | Needle Size |
| Cryomill | 9.43 ± 2 | 21 G |
| Cryogrind | 43.5 ± 14 | 21 G |
| Biopsy punch | 32.59 ± 6 | 21 G |
| (1.5 mm) | | |
| Meat grinder | 20.54 | 18 G |

As shown, the lowest injection force was created for the cryomilled product.

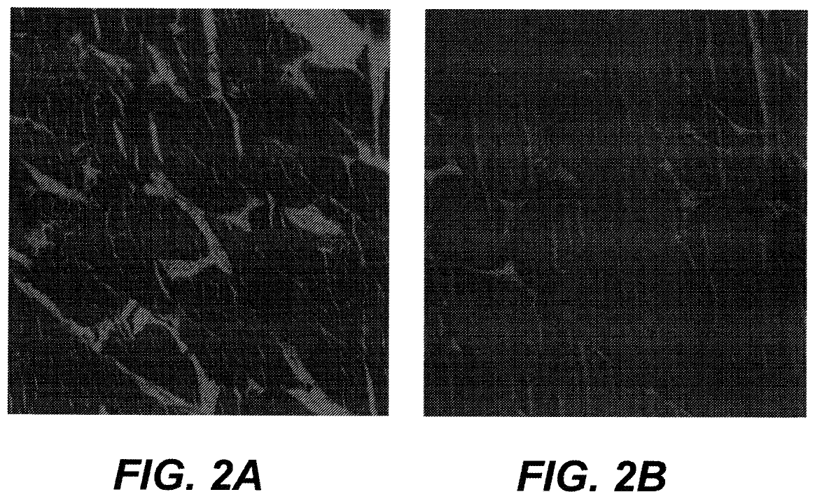

After processing, residual DNA content was analyzed, and DNA of the processed tissue was reduced to about 20 ng/mg. In addition, tissue samples were analyzed to confirm a cellularity. FIGS. 2A and 2B show fresh muscle as compared to processed muscle (biopsy punched samples). As shown, fresh muscle (sample A) retains nuclei (dark spots), whereas processed muscle (sample B) is acellular, as indicated by lack of nuclei.

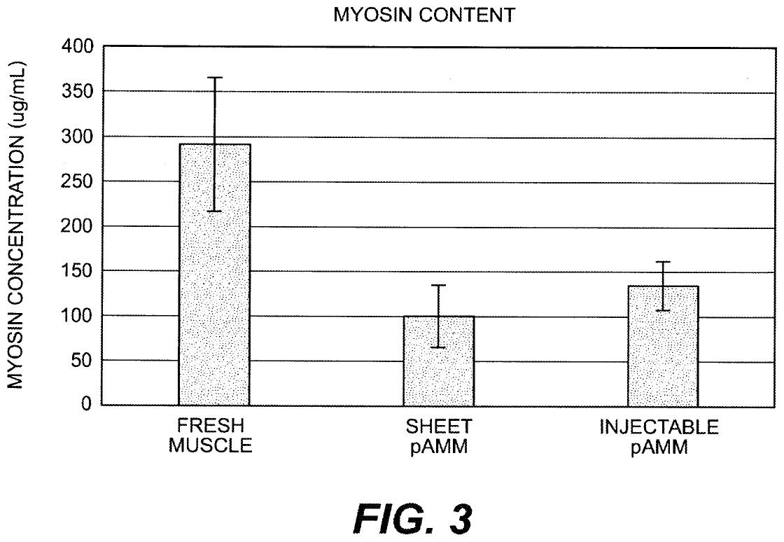

In addition, residual myosin content of pAMM in sheet or particulate form was analyzed with ELISA and compared to that of fresh muscle. As shown in FIG. 3, the myosin content of sheet and particulate muscle matrix was relatively similar and significantly lower than that of fresh muscle. The results indicate retention of myofibers needed to induce muscle formation.

### Example 2: Effect of Porcine Acellular Dermal Matrix (pADM) and (pAMM) on Muscle Regeneration

A defect was created in the rat rectus muscle by removing a piece of muscle measuring 15×5×5 mm (length×width×depth) in size. The defect was repaired with porcine acellular dermal matrix (pADM) (STRATTICE®) or a combination of pADM and sheet pAMM.

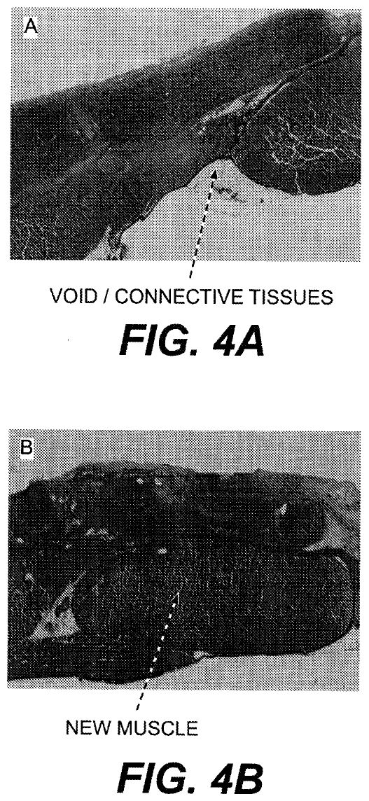

FIGS. 4A and 4B are trichome stained sections of rat rectus abdominal defects six weeks after creation and treatment with porcine acellular dermal matrix (FIG. 4A) or a combination of porcine acellular dermal matrix and porcine acellular muscle matrix (FIG. 4B). Minimal muscle regeneration was detected in defects repaired with only pADM. However, significant muscle regeneration was detected in defects repaired with a combination of pADM and pAMM 6 weeks after defect creation and repair. Connective tissues are stained blue and muscle fibers are stained red.

pAMM supports muscle regeneration in rats. Furthermore, only a muscle matrix is capable of supporting muscle regeneration.

### Example 3: Muscle Functional Recovery in Rat Models

To determine if pAMM-supported muscle regeneration translates into functional improvements, continuous running wheel and contraction force were studied. A defect in the gastrocnemius muscle of one leg of each rat was created by removing a piece of muscle approximately 10×8×4 mm (length×width×depth) in size and 20% by mass. The defects were either left unrepaired or repaired with sheet pAMM. The gastrocnemius muscle in the other leg of each animal was untouched and served as the contralateral control.

Running wheels equipped with an electronic counter connected to a computer interface were placed into the cages of the animals to monitor their voluntary activity and usage of the wheels after defect creation and repair. Additionally, muscle contractions were elicited in both the surgically affected and contralateral muscles by percutaneous electrical stimulation of the sciatic nerve that runs through the gastrocnemius muscle. The forces of the contractions were then measured.

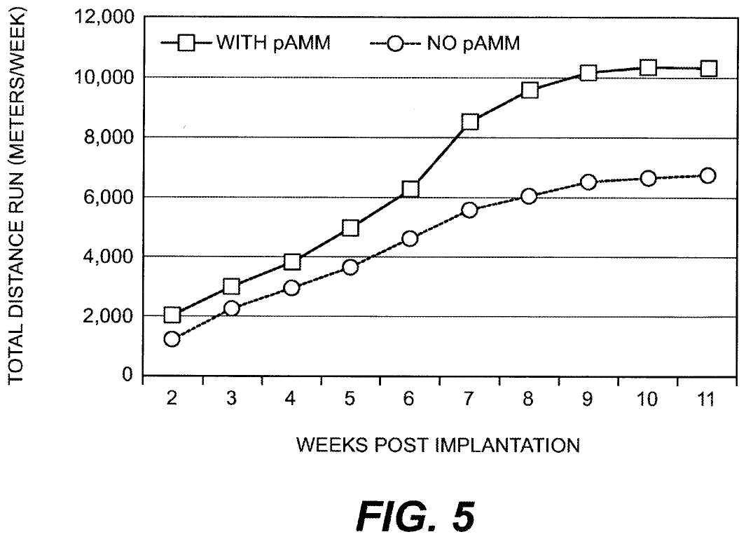

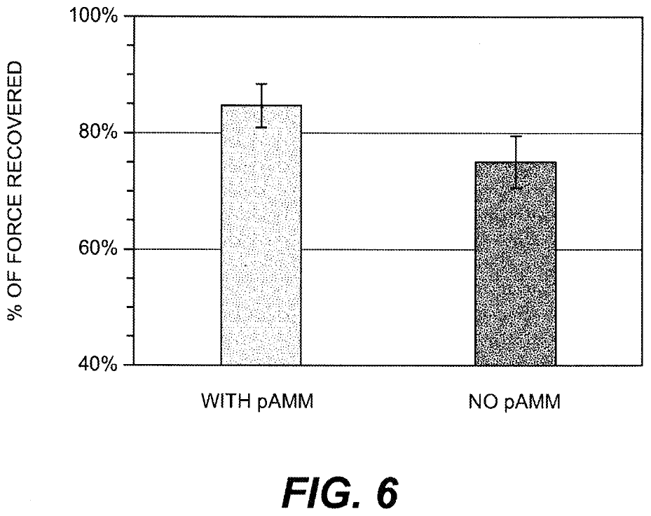

FIG. 5 is a graph of functional muscle recovery as measured by running distance for rats with gastrocnemius muscle defects left untreated or repaired with pAMM. FIG. 6 is a graph of functional muscle recovery as measured by percentage of contractile force exerted by rat muscles containing defects left unrepaired or repaired with pAMM compared to the force exerted by the contralateral rat muscles. Rats in which the defects were repaired with pAMM (With pAMM group) started running earlier and ran more than rats in which the defects were left unrepaired (No pAMM group). Furthermore, muscles in which defects were repaired with pAMM (With pAMM group) exerted more force than those with unrepaired defects. Notably, other studies provided evidence of innervation of the new muscle.

### Example 4: In Vivo Effect of Injectable Matrix in Rats with Damaged Muscle

Injectable muscle matrix prepared as described above was studied using a rat model. Particulate tissue was made using the meat grinder method. A defect was created in the tibialis anterior (TA) muscle in one leg of each rat by removing a piece of muscle 15×4×4 mm (length×width×depth) in size. The defects were either left unrepaired or repaired with injectable pAMM. The TA muscle in the other leg of each rat was untouched and served as the contralateral controls. After animals were sacrificed, the TA muscles from both legs were excised and evaluated for muscle weights and histological staining using hematoxylin and eosin (H&E) and Trichrome stains.

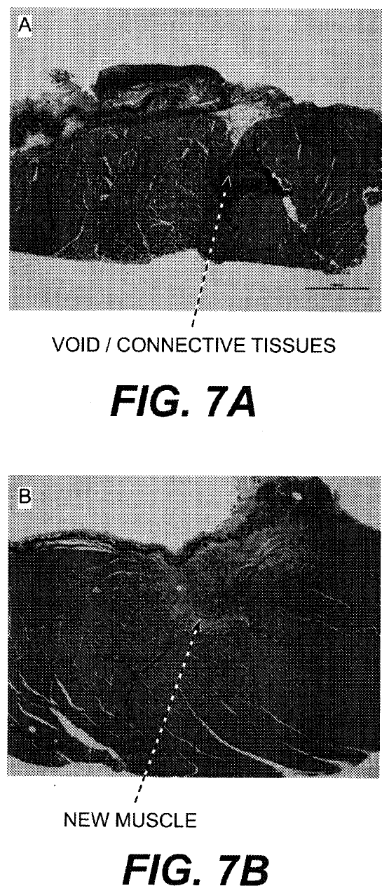

FIGS. 7A and 7B provide trichrome-stained images of rat tibialis anterior (TA) muscle defects three weeks after defect creation. Defects were either left unrepaired (A) or repaired with injectable pAMM (B) immediately after defect creation. Defects that were unrepaired were filled with connective, scar tissue with very little new muscle formation. Defects repaired with injectable pAMM demonstrated clear evidence of new muscle formation.

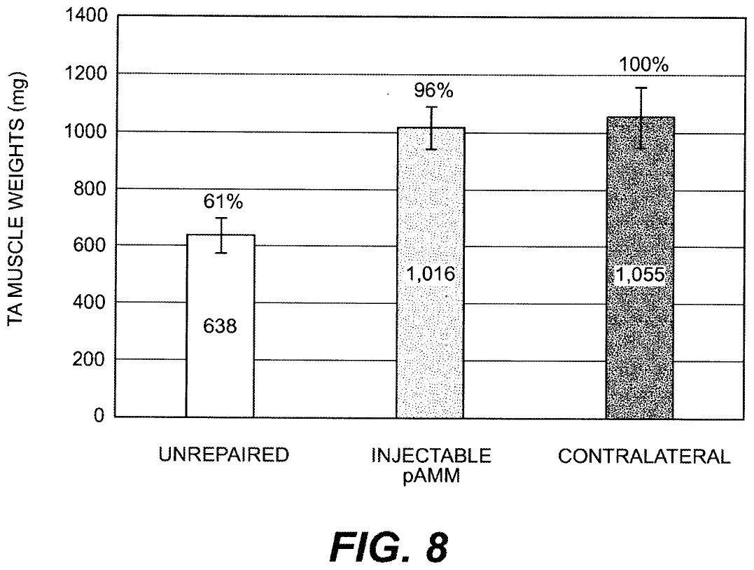

FIG. 8 is a bar graph illustrating TA muscle weights three weeks after defect creation with or without repair using injectable pAMM, and compared to the contralateral muscles. When measured, the weight of TA muscles that received pAMM weighed almost as much as the contralateral muscles while those with unrepaired defects weighed significantly less than the contralateral muscles. Accordingly, injectable pAMM supports muscle regeneration in a rat defect site. Percentages depicted in the graph represent the ratio of TA muscle weights in each group compared to the contralateral TA muscles.

### Example 5: In Vivo Effect of Injectable Matrix in Primates in Damaged Muscle

A defect in the gastrocnemius muscle of one leg of each primate (African green monkey) was created by removing a piece of muscle approximately 42×12×7 mm (length×width×depth) in size and 20% by mass. The defect was either left unrepaired (negative control group), repaired with sheet or injectable pAMM (made by hand chopping) or repaired with autologous minced primate muscle (positive control group).

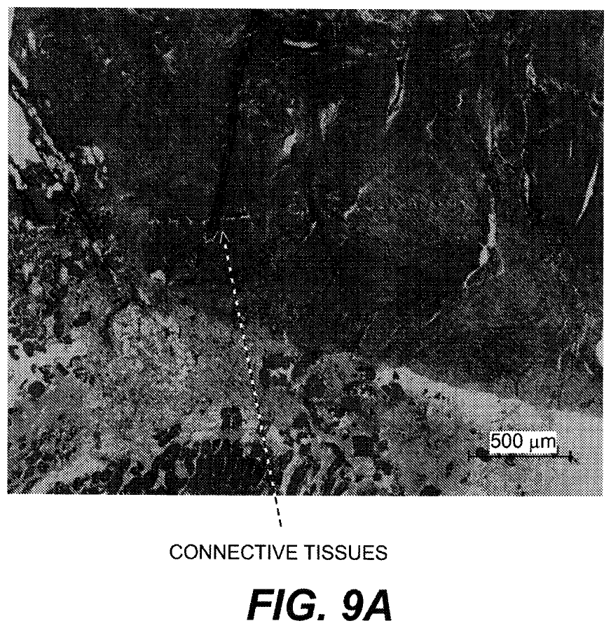

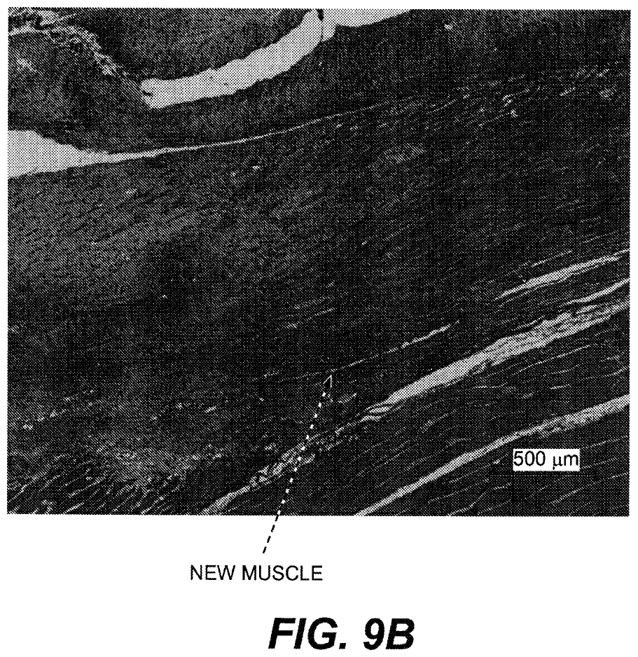

FIGS. 9A and 9B are trichrome-stained images of primate gastrocnemius defects twelve weeks after defect creation. Defects were either left unrepaired (A) or repaired with pAMM (B) immediately after defect creation. Results of the study showed that minimal muscle regeneration occurred in defects left untreated. The defect site was filled with connective scar tissue. In contrast, multiple new muscle bundles were detectable in defects repaired with either form of pAMM (sheet or injectable). In some cases, the degree of regeneration was similar to those seen in defects repaired with autologous minced primate muscle. pAMM can support muscle regeneration in primates.

### Example 6: In Vivo Effect of Injectable Matrix in Rats with Non-Injured Muscle to Support New Muscle Formation

Cryomilled injectable porcine acellular muscle matrix (pAMM) prepared as described in Example 1 was injected into a normal tibialis anterior (TA) muscle in the right leg of each rat (no defects existed or were created). The TA muscle in the left leg did not receive any pAMM and served as the contralateral control for each animal. Animals were sacrificed at 1 week, 2 weeks, and 3 weeks. The TA muscles from both the injected and contralateral legs were excised and evaluated for gross observations, muscle weights and histological staining using hematoxylin and eosin (H&E) and Trichrome stains.

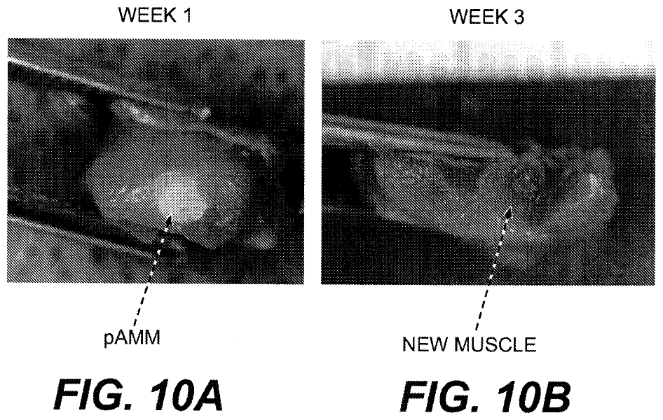

FIGS. 10A and 10B provide gross cross-sectional images of rat TA muscles injected with pAMM at one or three weeks after injection. The implanted pAMM was easily visible at the 1 week time point but decreased throughout the study period. At the 3 week time point, no pAMM was detectable by the naked eye but residual amounts were detectable histologically.

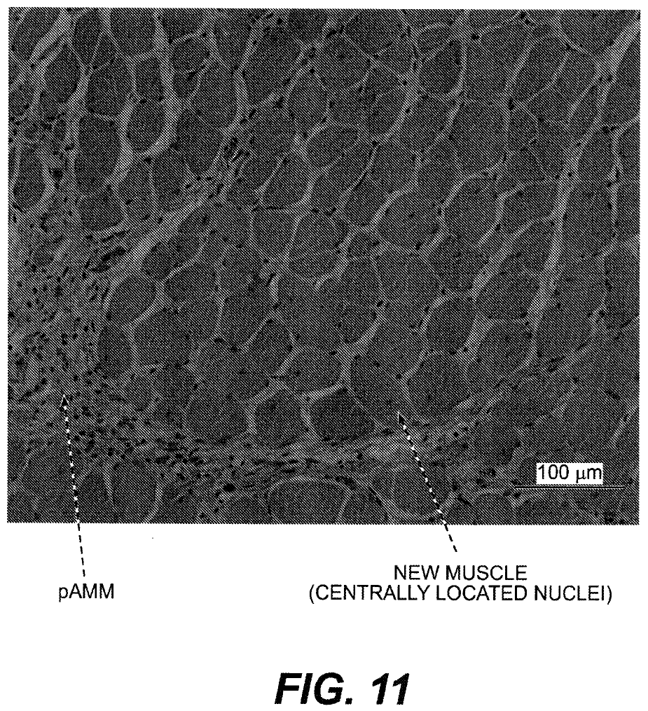

Furthermore, evaluation of Trichrome and H&E stained explants revealed evidence of new myofibers at all time points examined. FIG. 11 depicts a H&E stained cross section image of pAMM injected TA muscle at 3 weeks post implantation. At three weeks, large bundles of new myofibers similar in size to those in the native muscle were detectable, especially in the area surrounding the residual pAMM. Many of the new myofiber bundles contained centrally located nuclei, a trait characteristic of newly formed and maturing bundles.

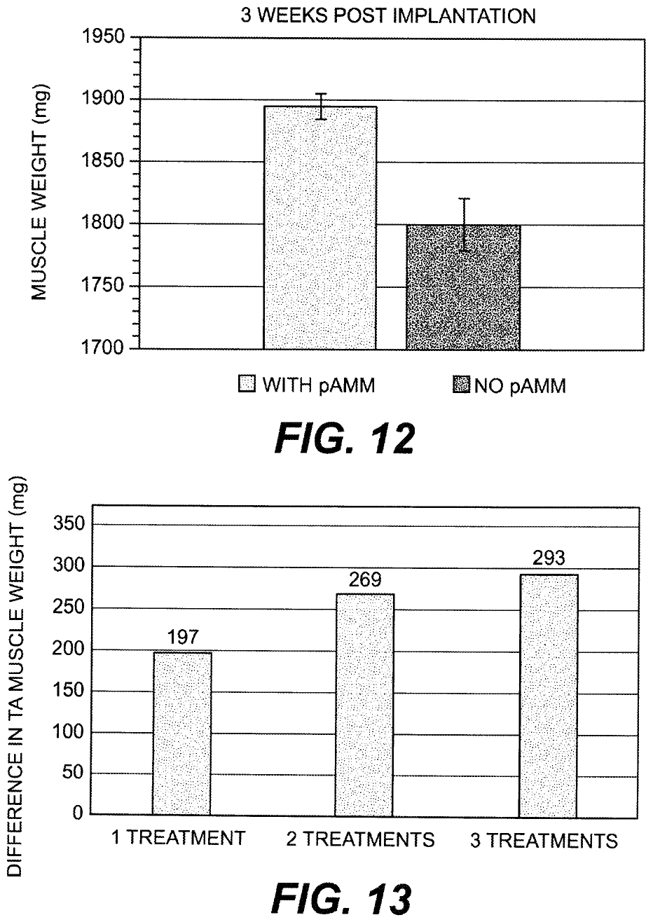

At three weeks, TA muscles that received pAMM weighed more than the contralateral muscles. FIG. 12 is a bar graph of rat TA muscles with or without pAMM injection three weeks after treatment to show pAMM ability to augment existing muscle mass. Since there was no detectable scar tissue (grossly or histologically) and only residual amounts of pAMM detectable, the extra weight can only be attributed to new muscle. Injectable pAMM supports muscle formation (regeneration) in a rat non-injured muscle.

### Example 7: Study of Repeat Treatments

An additional study was performed to determine if repeat treatment with pAMM increases the amount of new muscle formation.

Rats were divided into three groups. In all groups, pAMM (porcine acellular muscle matrix) was injected into a normal TA muscle in the right legs of rats. The TA muscles in the left legs did not receive any injections and served as the contralateral control. Animals in each of the three groups received different numbers of treatments at different timepoints as follows:

Group 1—animals received pAMM injections at T=0

Group 2—animals received pAMM injections at T=0 and at T=3 weeks

Group 3—animals received pAMM injections at T=0, at T=3 weeks and at T=6 weeks

131+/−15 mg (average+/−stdev) of pAMM was injected into an animal at each treatment. All animals were sacrificed 9 weeks after the first treatment at T=0.

After 9 weeks, muscles that received 1 treatment weighed an average of 197 mg more than their contralateral muscles. However, muscles that received 3 treatments weighed an average of 292 mg more than their contralateral muscles (FIG. 13).

The average weight difference between the injected and contralateral TA muscles increases with increasing numbers of treatments, as illustrated in FIG. 13, which is a bar graph depicting weight differences between injected and contralateral TA muscles at 9 weeks after the first treatment.

### Example 8: Effect of Particle Size on Volume Retention

It is recognized that larger matrix particles may not be as easy to inject (i.e., will require a larger needle or higher injection force). However, the effect of particle size on matrix volume retention is not entirely clear. Accordingly, injections were made using differing particle sizes.

Accordingly, the purpose of this study was to determine if pAMM particle size affects (1) the kinetics of pAMM in vivo retention or (2) the degree of new muscle formation

Rats were divided into four groups. In all groups, pAMM (porcine acellular muscle matrix) was injected into a normal TA muscle in the right legs of rats. The TA muscles in the left legs did not receive any injections and served as the contralateral control. Animals in each group received different materials as follows:

Group 1—animals received cryoground pADM

Group 2—animals received cryomilled pAMM-˜100 μm particle size (Formulation 1)

Group 3—animals received cryoground pAMM-˜600 μm particle size (Formulation 2)

Group 4—animals received biopsy punched pAMM-˜1.5 mm particle size (Formulation 3)

Approximately 150 mg of material was injected into each animal. For each group, half of the animals were sacrificed 3 weeks after injection and half of the animals were sacrificed 6 weeks after injection.

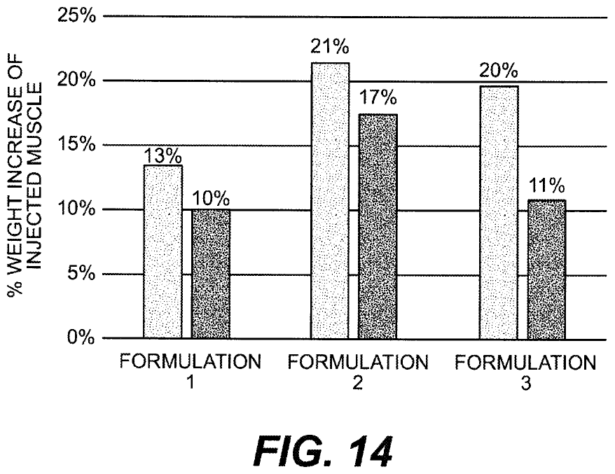

FIG. 14 is a bar graph depicting the percentage weight increase of injected muscle compared to their corresponding untreated contralateral muscle. Muscles injected with Formulation 1 weighed 22% and 10% more than their contralateral muscle at 3 weeks and 6 weeks respectively. (The 13% difference reported in the graph for Formulation 1 at 3 weeks is the average of the values from this study and the values from the 3 week time point from another study that also used Formulation 1 material). Muscles injected with Formulation 2 weighed 21% and 17% more than their contralateral muscles at 3 weeks and 6 weeks, respectively. Muscles injected with Formulation 3 weighed 20% and 11% more than their contralateral muscles at 3 weeks and 6 weeks, respectively.

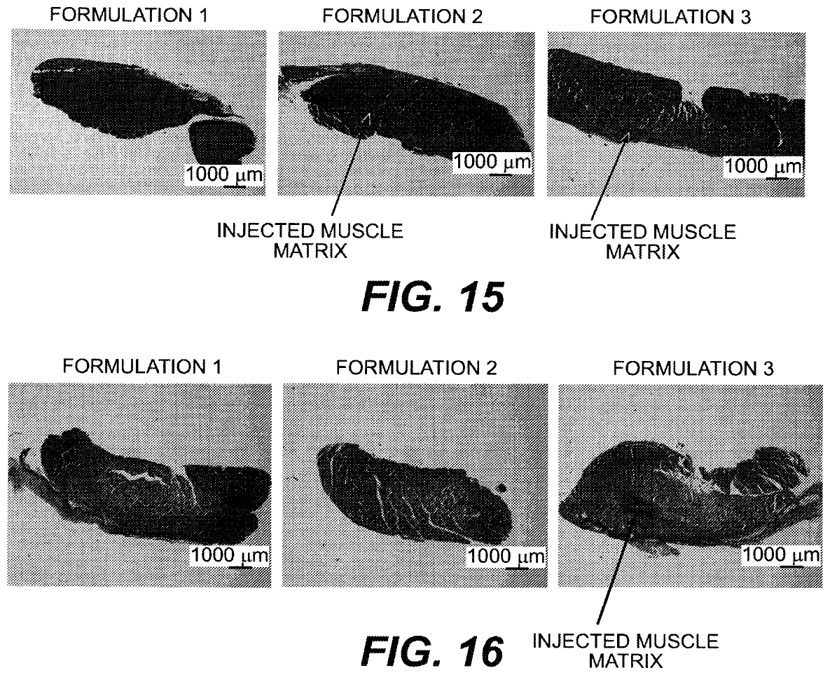

Trichrome stained images of muscles injected with Formulation 1 showed that no residual pAMM was detectable 3 weeks after injection, whereas trichrome stained images of muscles injected with Formulations 2 & 3 show small but detectable amounts of residual pAMM 3 weeks after injection (FIG. 15). No residual pAMM of either Formulation 1 or Formulation 2 was detectable 6 weeks after injection. Minimal amounts of Formulation 3 were still detectable 6 weeks after injection (FIG. 16).

In vivo retention of pAMM is inversely proportional to particle size with larger particles being retained longer than smaller particles. However, Formulation 2 pAMM injections resulted in the largest weight difference between injected and contralateral muscles at both 3 weeks and 6 weeks post-injection.

### Example 9: Effect of Trypsin Concentration on Myofiber Retention

It is recognized that altering the concentration of trypsin applied to skeletal muscle will affect the processing of the skeletal muscle, particularly the retention of myofibers. Accordingly, different concentrations of trypsin were used, and the uniformity of processed samples, both within each piece and compared to the others, was studied. The uniformity of the tissue was determined by visual inspection at the end of all processing steps.

Porcine muscle tissue was sliced into 5 cm by 10 cm by 5 mm sizes. The tissue samples were then frozen, cut, treated with a protective solution, treated with trypsin, decellularized, washed with PBS, and stored in a refrigerator. Once cut, the tissue samples were treated at room temperature. The control tissue was not treated with trypsin and instead sat in PBS for an additional time period.

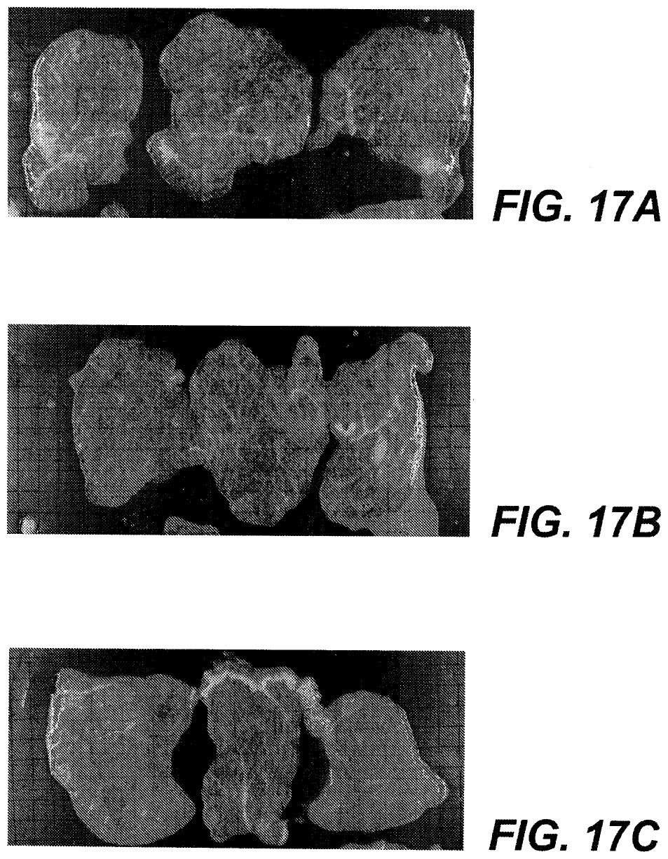

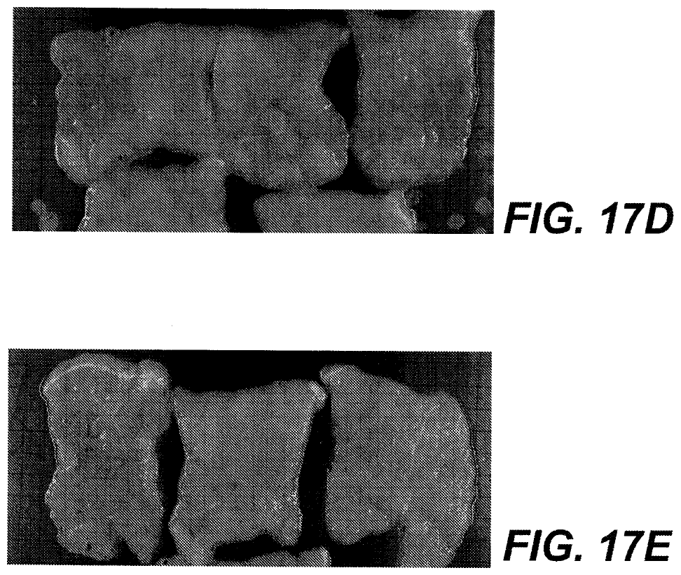

FIG. 17A depicts tissue samples treated with trypsin at a concentration of 10⁻⁴ weight by volume. FIG. 17B depicts tissue samples treated with trypsin at a concentration of 10⁻⁵ weight by volume. FIG. 17C depicts tissue samples treated with trypsin at a concentration of 10⁻⁶ weight by volume. FIG. 17D depicts tissue samples treated with trypsin at a concentration of 10⁻⁷ weight by volume. FIG. 17E depicts tissue samples treated with trypsin at a concentration of 10⁻⁸ weight by volume.

The outcome of the study was determined by visual inspection of the decellularized tissue and differential scanning calorimetry measurement at the end of processing. In this study, it appears that the range of trypsin concentrations explored were suitable in terms of being able to control the retention of myofibers within the porcine tissue. The final tissue appearances were uniform within each piece and consistent from piece to piece. At high concentrations (starting at 10⁻⁴), the tissue developed mesh-like features. At concentrations in the 10⁻⁷ and 10⁻⁸ range, the processed muscle tissue appeared to be able to retained most of its myofibers at the end of the experimental process.

### Example 10: Effect of Trypsin Concentration and Treatment Time on Myosin Retention

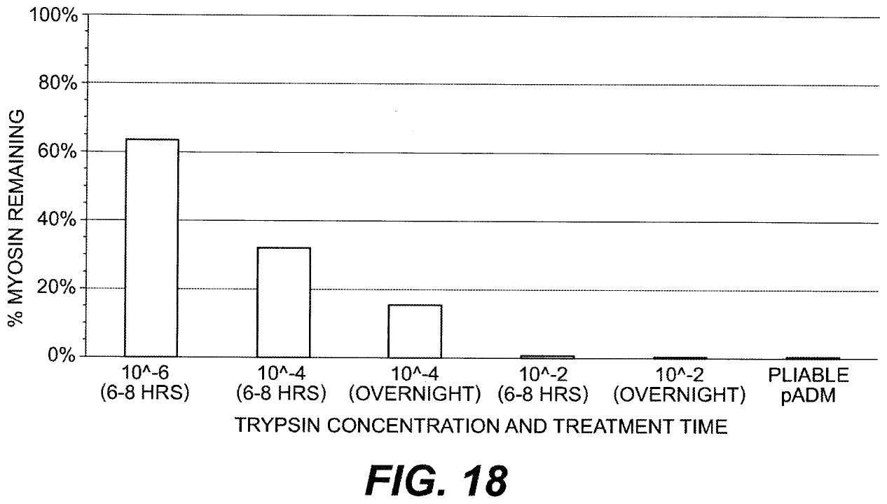

The study described in Example 9 was repeated at different trypsin concentrations for varying amounts of time. FIG. 18 depicts the myosin concentrations measured after trypsin treatment.

Trypsin treatment at a concentration of 10⁻⁶ weight by volume for 6-8 hours produced a tissue that retained a 40-60% myosin concentration of that of fresh unprocessed muscle tissue. Tissue treated with a trypsin concentration of 10⁻⁴ weight by volume for 6-8 hours retained more myosin as compared to tissue treated with the same trypsin concentration overnight but less than the tissue treated with a concentration of 10⁻⁶ weight by volume for 6-8 hours. These results suggests that myosin concentration is time and enzyme concentration dependent.

### Example 11: Effect of Trypsin and Bromelain on Myofiber Retention

Porcine muscle tissue samples of 8 mm and 5 mm were treated as described in Example 9 with a trypsin concentration of 10⁻² or 10⁻⁴ weight by volume. Some samples were treated with bromelain at a concentration of 10 units per liter (U/L). Trypsin treated samples were treated at temperatures of 4° C. or room temperature. Some trypsin treated samples were cut along a cross-section and others were cut along a longitudinal section.

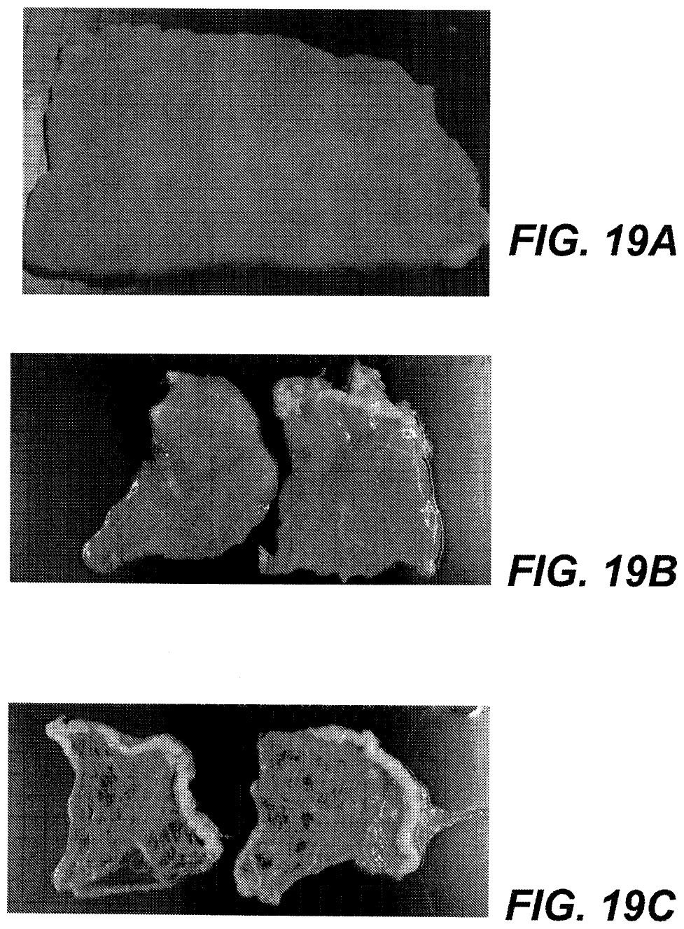

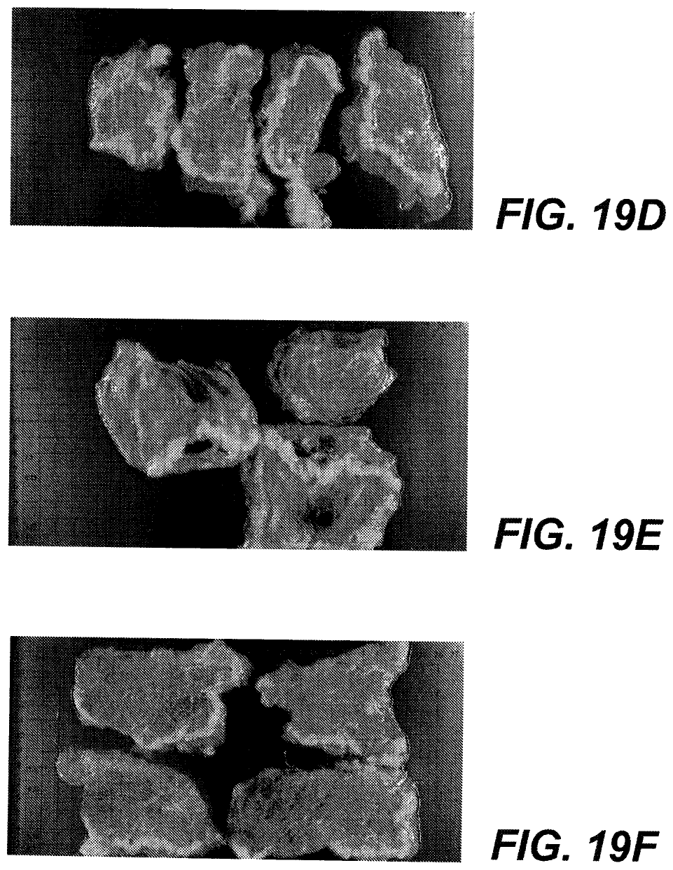

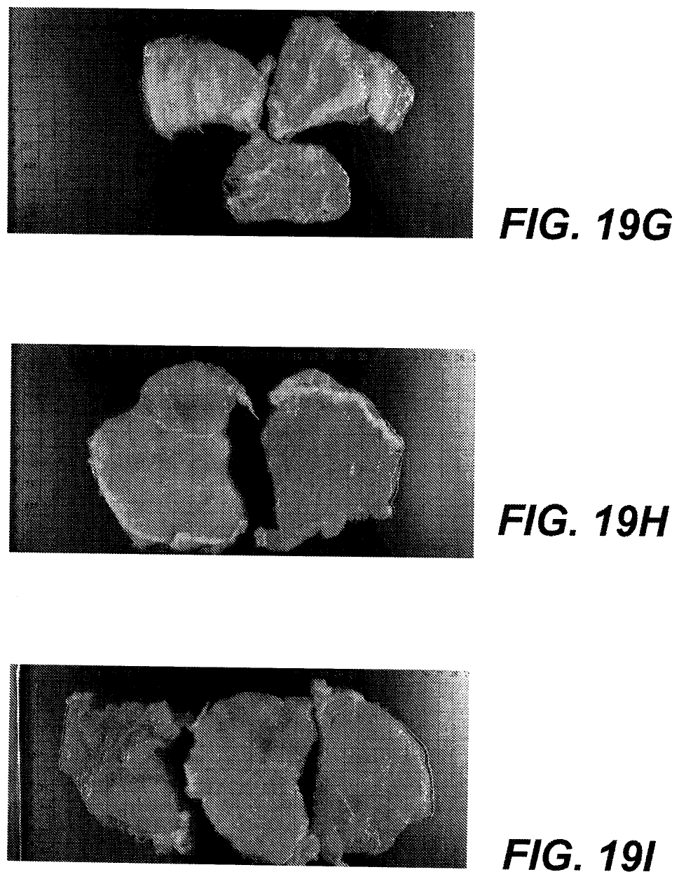

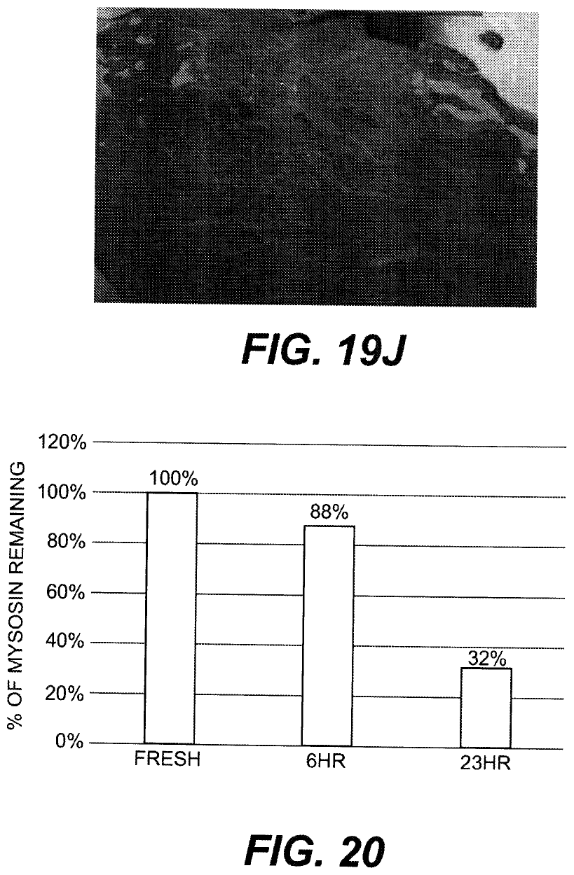

FIG. 19A depicts a control tissue that was not treated with trypsin or bromelain. FIG. 19B depicts porcine muscle tissue of 8 mm thickness treated with a trypsin concentration of 10⁻⁴ weight by volume at room temperature. FIG. 19C depicts porcine muscle tissue of 5 mm thickness treated with a trypsin concentration of 10⁻⁴ weight by volume at room temperature. FIG. 19D depicts a cross-section of porcine muscle tissue treated with a trypsin concentration of 10⁻² weight by volume at room temperature. FIG. 19E depicts a longitudinal-section of porcine muscle tissue treated with a trypsin concentration of 10⁻² weight by volume at room temperature. FIG. 19F depicts a cross-section of porcine muscle tissue treated with a trypsin concentration of 10⁻² weight by volume at a temperature of 4° C. FIG. 19G depicts a longitudinal-section of porcine muscle tissue treated with a trypsin concentration of 10⁻² weight by volume at a temperature of 4° C. FIG. 19H depicts porcine muscle tissue of 5 mm thickness treated with 10 U/L of bromelain at room temperature. FIG. 19I depicts porcine muscle tissue of 8 mm thickness treated with 10 U/L of bromelain at room temperature. FIG. 19J depicts porcine muscle tissue treated with a 100 U/L of bromelain at room temperature.

Porcine tissue samples were evaluated by visual inspection at the end of processing. The control tissue processed without the use of trypsin exhibited very little myofiber loss during processing. The control tissue appeared to be dense and opaque in color. The tissue treated with the 10⁻² trypsin concentration exhibited a demonstrable loss of myofibers, leaving behind a mesh like tissue network. This was true whether the tissue was processed in the cross section or longitudinal direction, and true at both 4° C. or room temperature. When using 10⁻⁴ trypsin concentration, the 8 mm thick muscle tissue showed a much greater myofiber retention over the 5 mm thick muscle tissue. The 10 U/L bromelain solution, on the other hand, appeared to have much less of an effect on removing the myofibers. Tissue treated with 100 U/L of bromelain retained 88% of myofibers after 6 hours of treatment and 32% of myofibers after 23 hours of treatment, as shown in FIG. 20.

The preceding examples are intended to illustrate and in no way limit the present disclosure. Other embodiments of the disclosed devices and methods will be apparent to those skilled in the art from consideration of the specification and practice of the devices and methods disclosed herein.

1. A method of preparing a muscle implant, comprising:
providing at least one muscle sample;
contacting the at least one muscle sample with a solution containing trypsin;
decellularizing the at least one muscle sample to produce at least one decellularized muscle matrix as measured with light microscopy; and
processing the decellularized muscle matrix to produce a collection of decellularized muscle matrix particles between about 3 microns and 5000 microns, wherein the collection of particles are capable of supporting regeneration of muscle tissue, and wherein the contacting with a solution containing trypsin and the decellularizing are controlled in order to retain in the collection of decellularized muscle matrix particles about 10-60% of the myosin normally found in the muscle sample prior to processing.

2. The method of claim 1, wherein the at least one muscle sample is at least two or at least three muscle samples.

3. The method of claim 1, wherein the at least one muscle sample is decellularized by contacting the sample with a decellularization solution comprising at least one of a polyethylene glycol, sodium dodecyl sulfate, sodium deoxycholate, and polyoxyethylene (20) sorbitan monolaurate.

4. The method of claim 1, wherein controlling the contacting with a solution containing trypsin comprises controlling the exposure duration and concentration of the trypsin solution.

5. The method of claim 1, further comprising contacting the at least one muscle sample with DNase.

6. The method of claim 1, further comprising contacting the at least one muscle sample with alpha-galactosidase.

7. The method of claim 1, wherein the at least one muscle sample is from an animal that lacks substantially all alpha-galactose moieties.

8. The method of claim 1, wherein processing the decellularized muscle matrix to produce the collection of decellularized muscle matrix particles comprises blending, cutting, or cryofracturing the muscle matrix.

9. (canceled)

10. (canceled)

11. The method of claim 1, wherein the trypsin is at a concentration ranging from 10⁻⁸% weight by volume to 10⁻⁴% weight by volume.

12. The method of claim 11, wherein the trypsin is at a concentration ranging from 10⁻⁷% weight by volume to 10⁻⁴% weight by volume.

13. The method of claim 1, wherein the collection of decellularized muscle matrix particles retains about 20-30% of the myosin normally found in an unprecessed muscle sample.

14. The method of claim 1, wherein processing the decellularized muscle matrix to produce the collection of decellularized muscle matrix particles produces a collection of decellularized muscle matrix particles between about 100 microns and 800 microns.

15. The method of claim 1, wherein processing the decellularized muscle matrix to produce the collection of decellularized muscle matrix particles produces a collection of decellularized muscle matrix particles between about 50 microns and 200 microns.

16. A muscle implant including a collection of muscle matrix particles capable of supporting regeneration of muscle tissue, the particles being between about 3 microns and 5000 microns and comprising trypsin treated decellularized muscle matrix that contains about 10-60% of the myosin normally found in an unprocessed muscle sample.

17. The muscle implant of claim 16, wherein the muscle matrix contains about 20-30% of the myosin normally found in an unprocessed muscle sample.

18. The muscle implant of claim 16, wherein the decellularized muscle matrix lacks substantially all alpha-galactose moieties.

19. The muscle implant of claim 16, wherein the muscle implant is lyophilized or in aqueous solution.

20. (canceled)

21. The muscle implant of claim 16, wherein the decellularized muscle matrix has been treated with trypsin at a concentration ranging from 10⁻⁸% weight by volume to 10⁻⁴% weight by volume.

22. A method of treatment, comprising injection into a patient a muscle implant, wherein the muscle implant comprises particulate decellularized muscle matrix containing at least some of the myofibers normally found in an unprocessed muscle sample.

23. The method of claim 18, wherein the at least one decellularized muscle matrix contains about 10-60% of the myofibers normally found in an unprocessed muscle sample.
